(19) United States
(12) Patent Application Publication
(10) Pub. No.: US 2015/0246141 A1
(43) Pub. Date: Sep. 3, 2015

(54) DIAGNOSTIC AGENTS WITH ENHANCED SENSITIVITY/SPECIFICITY

(71) Applicant: BEN-GURION UNIVERSITY OF THE NEGEV RESEARCH AND DEVELOPMENT AUTHORITY, Beer Sheva (IL)

(72) Inventor: Ayelet David, Omer (IL)

(73) Assignee: Ben-Gurion University of the Negev Research and Development Authority, Beer Sheva (IL)

(21) Appl. No.: 14/427,946

(22) PCT Filed: Sep. 12, 2013
(86) PCT No.: PCT/IL2013/050779

§ 371 Date: Mar. 12, 2015

Related U.S. Application Data
(60) Provisional application No. 61/700,367, filed on Sep. 13, 2012.

(51) Int. Cl. A61K 49/00; C08F 122/36

(52) U.S. Cl. CPC A61K 49/0054; A61K 49/0032; C08F 122/36

(57) ABSTRACT

Highly sensitive imaging of diseased tissues such as cancer is attractive because it potentially allows for early tumor detection. One of the problems associated with conventional, low molecular weight imaging probes is the limited tumor: background ratio. To circumvent this, imaging probes were conjugated to polymeric carriers and were surprisingly found to accumulate specifically at cancer sites. This invention describes an innovative targeting strategy for the selective identification of solid tumors by means of polymer-NIR fluorochrome conjugates which accumulate selectively within cancerous tissue relative to normal tissue.

### FIELD OF THE INVENTION

This invention describes polymer-chromophore conjugates optionally comprising a solubilizing agent and methods of use thereof as diagnostic agents, which exhibit enhanced specificity and/or sensitivity.

### BACKGROUND OF THE INVENTION

Optically based biomedical imaging techniques have advanced over the past decade due to factors including developments in laser technology, sophisticated reconstruction algorithms and imaging software originally developed for non-optical, tomographic imaging modes such as CT and MRI. Visible wavelengths are used for optical imaging of surface structures by means of endoscopy and microscopy.

Near infrared wavelengths (approx. 700-1000 nm) have been used in optical imaging of internal tissues, because near infrared radiation exhibits tissue penetration of up to 6-8 centimeters. See, e.g., Wyatt, 1997, “Cerebral oxygenation and haemodynamics in the fetus and newborn infant,” Phil. Trans. R. Soc. London B 352:701-706; Tromberg et al., 1997, “Non-invasive measurements of breast tissue optical properties using frequency-domain photo migration,” Phil. Trans. R. Soc. London B 352:661-667.

Advantages of near infrared imaging over other currently used clinical imaging techniques include the following: potential for simultaneous use of multiple, distinguishable probes (important in molecular imaging); high temporal resolution (important in functional imaging); high spatial resolution (important in in vivo microscopy); and safety (no ionizing radiation).

In near infrared fluorescence imaging, filtered light or a laser with a defined bandwidth is used as a source of excitation light. The excitation light travels through body tissues. When it encounters a near infrared fluorescent molecule (“contrast agent”), the excitation light is absorbed. The fluorescent molecule then emits light (fluorescence) spectrally distinguishable (slightly longer wavelength) from the excitation light. Despite good penetration of biological tissues by near infrared light, conventional near infrared fluorescence probes are subject to many of the same limitations encountered with other contrast agents, including large volume of distribution and low target/background ratios.

There remains a need for effective targeting of cancerous cells and tissue and thereby an effective cancer diagnostic and others.

### SUMMARY OF THE INVENTION

In one embodiment this invention provides a polymer characterized by the structure of polymer characterized by the structure of formula 1:

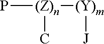

wherein
m, n and indicate percentages of the respective monomer composition of the polymer, wherein m is between about 0%-50%, n is between 0.05 to 50%;
C is a near infrared dye selected from the group consisting of Cy5, Cy5.5, Cy7,
Indocyanine green (ICG), IR783 and analogs thereof, covalently linked to the polymeric backbone and present in a concentration of between about 0.5 to 50%.
Y is a spacer arm linking J to the polymeric backbone, wherein said spacer arm is an alkane, alkene or a peptidic chain of 6 to 18 atoms;
Z is a spacer arm linking C to the polymeric backbone, wherein said spacer arm is an alkane, alkene or a peptidic chain of 6 to 18 atoms
J is a solubilizing agent and present in a concentration of between about 0 to 50%, and
P is a polymeric backbone comprising underivatized or derivatized N-(2-hydroxypropyl)methacrylamide (HPMA) monomers of, underivatized or derivatized N-methylacrylamide monomers, underivatized or derivatized N,N-dialkylacrylamides monomers, underivatized or derivatized acrylic acid, underivatized or derivatized methacrylic acid underivatized or derivatized polyamino acids, underivatized or derivatized polysaccharides, underivatized or derivatized polymers containing polyethyleneoxide sequences and polyvinyl pyrrolidone-maleic anhydride polymers, underivatized or derivatized polylactic-co-glycolic acid, underivatized or derivatized dendrimers, underivatized or derivatized polysaccharides, underivatized or derivatized peptides, underivatized or derivatized proteins, underivatized or derivatized polymer-peptide conjugates or underivatized or derivatized polymer-protein conjugates or mixed polymers.

In one embodiment, this invention provides a polymer represented by the structure of formula III:

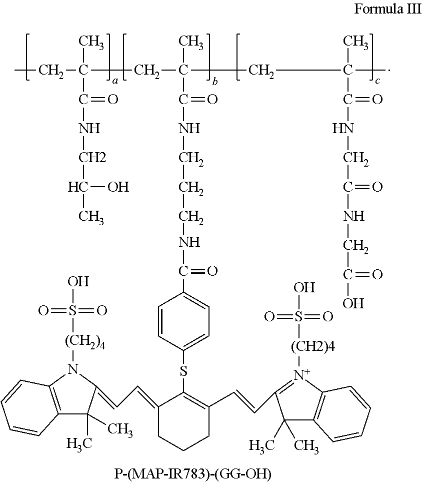

In some embodiments, the invention provides a diagnostic composition comprising a polymer of this invention. In some embodiments, such composition is specifically formulated for intraluminal or mucosal administration.

In some embodiments, the invention provides a method of imaging an inflammatory condition in a subject, said method comprising administering a polymer of this invention to said subject.

In some embodiments, the invention provides a method of imaging a disease associated with neovascularization in a subject, said method comprising administering a polymer of this invention to said subject.

In some embodiments, the invention provides a method of imaging a cancer or cancerous tissue in a subject, said method comprising the step of contacting said cancer or cancerous tissue with a polymer of this invention.

In some embodiments, the method comprises administering the polymer intraluminally to a gastrointestinal tract surface.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts the MALDI-TOF mass spectrometry results showing peaks at 845.3, 867.2 and 883.2, calculated for M+H+, M+Na+, and M+K+, respectively, of an IR-783-S-Ph-COOH synthesis.

FIG. 2 schematically depicts the synthetic scheme for the synthesis of an HPMA copolymer bearing IR783-S-Ph-COOH (P-(AP)-IR783)

FIG. 3 depicts selective accumulation of P-(AP-IR783) in cancerous tissue occurred when the polymer was applied intraluminally to mice. P-(AP-IR783) application to control mice having no tumors exhibited minimal background staining (FIG. 3A-C), whereas distinct staining was evident in samples taken from mice having tumors (verified by pathologic evaluation subsequently) (FIGS. 3D-G). Tumor associated-vasculature staining was evident, as well (FIG. 3F).

FIG. 4 depicts selective accumulation of P-(AP-IR783) in cancerous tissue when the polymer was applied intraluminally to mice, when visualized only 2 hours after the wash (FIG. 4A), or immediately post administration of 0.1 mg/ml (200 μg) P-(AP-IR783), where surface exposed cells (boxes 2, 3) were stained intensely, but whereas region 4 did not evidence the presence of tumors when viewed macroscopically, the region stained intensely, and proved to contain tumors in the submucosa (FIG. 4B), in mouse HT-29 models. FIGS. 4C and 4D provide results of similarly treated animals as in FIG. 4B, 2 hours post-administration. The phenomenon of evident staining of both surface exposed (T) and submucosal tumors (N) is maintained even two hours after exposure, indicating retention/accumulation of the marker in cancerous tissue.

FIG. 5 presents selective markedly intense staining of applied conjugate polymer at cancerous regions in the tissue (polyp and tumor) with very low binding to near healthy tissue.

FIG. 6 depicts selective accumulation of P-(AP-IR783) in cancerous tissue with intraluminal polymer application to various tissues in mice. 40 μg per mouse of P-(AP-IR783) administered intraluminally, provided intense staining in the excised mouse colon tumor tissue, stomach, liver, lungs and feces (FIG. 6A), with very little background staining is seen in unaffected tissues and samples (e.g. heart, small bowel, urine) (images taken at 3 hours following washing). The table in FIG. 6B plots the significance of these findings in terms of the Em value obtained at 0.05, 0.5 and 2 seconds post administration showing staining over time in the stomach, liver and lungs.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This invention provides, inter alia, for the specific targeting of imaging agents. In one embodiment this invention provides a polymer characterized by the structure of formula

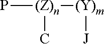

wherein
m, n and indicate percentages of the respective monomer composition of the polymer, wherein m is between about 0%-50%, n is between 0.05 to 50%;
C is a near infrared dye selected from the group consisting of Cy5, Cy5.5, Cy7,
Indocyanine green (ICG), IR783 and analogs thereof, covalently linked to the polymeric backbone and present in a concentration of between about 0.5 to 50%.
Y is a spacer arm linking J to the polymeric backbone, wherein said spacer arm is an alkane, alkene or a peptidic chain of 6 to 18 atoms;
Z is a spacer arm linking C to the polymeric backbone, wherein said spacer arm is an alkane, alkene or a peptidic chain of 6 to 18 atoms
J is a solubilizing agent and present in a concentration of between about 0 to 50%, and
P is a polymeric group comprising underivatized or derivatized monomers of N-(2-hydroxypropyl)methacrylamide (HPMA), underivatized or derivatized monomers of N-methylacrylamide, underivatized or derivatized monomers of N,N-dialkylacrylamides, underivatized or derivatized acrylic acid, underivatized or derivatized methacrylic acid polyamino acids, underivatized or derivatized polysaccharides, underivatized or derivatized polymers containing polyethyleneoxide sequences and polyvinyl pyrrolidone-maleic anhydride polymers, underivatized or derivatized polylactic-co-glycolic acid, dendrimers, underivatized or derivatized peptides, underivatized or derivatized proteins, underivatized or derivatized polymer-peptide conjugates or underivatized or derivatized polymer-protein conjugates or mixed polymers.

In one embodiment the invention provides a polymer of formula 1 wherein the molecular weight of the polymer ranges between 100 Da and 1000 kDa. In one embodiment the molecular weight of the polymer is less than 60 kDa. In one embodiment, the molecular weight of the polymer ranges between 15-60 kDa. It will be appreciated by the skilled artisan that molecular weight may vary as a function of the particular monomers chosen, and that such variations are to be considered as part of this invention.

In one embodiment the composition comprises a polymer of formula 1 containing about 60-80 molar % of P and about 20-40 molar % of C and when J is present, from about 0.5-20 molar % of J. In some embodiments, the polymer contains from about 0.5-40 molar % of Z. In some embodiments, when J is present, the polymer contains about 0.5-40 molar % of Z and 0.5-20 molar % of Y.

In one embodiment Y or Z is characterized by the structure of formulae IIa, or IIb or IIc as follows:

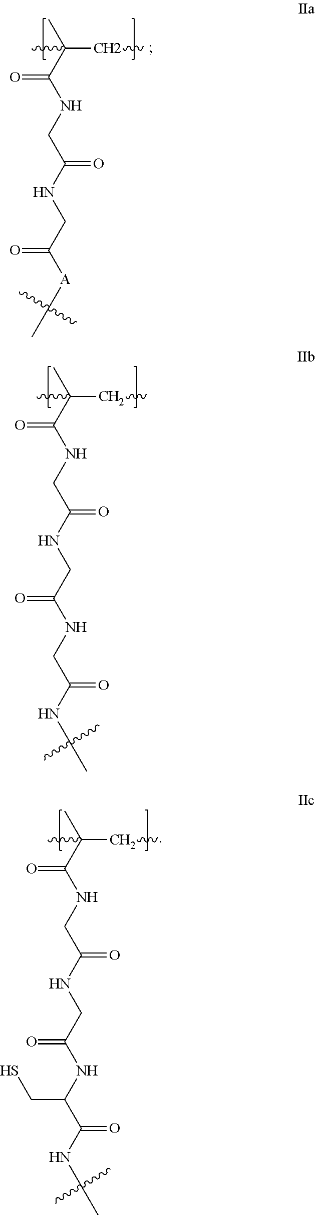

In some embodiments, Y or Z is Gly-Gly.

In one embodiment the polymer is represented by the structure of formula III:

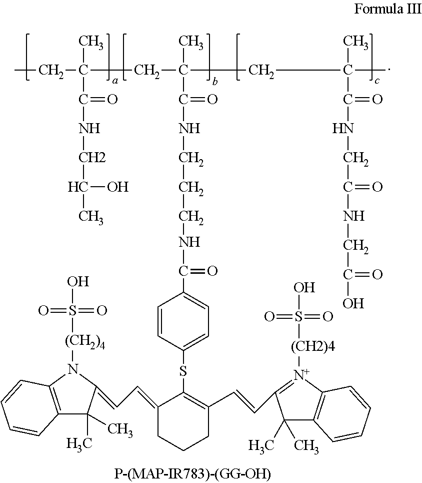

Use of the described polymer conjugates of this invention for diagnostic applications for tumor/cancer identification tissues is surprisingly associated with a limited tumor-to-background ratio.

In one embodiment of this invention, despite the reported phenomenon of passive accumulation of macromolecules into tumor tissues due to the “enhanced permeability and retention” effect (EPR effect), the polymers as herein described provide superior results in terms of their detection sensitivity, as compared to other systems incorporating conjugated polymers containing various dyes. In one embodiment, this invention provides a highly sensitive diagnostic method which can, in turn, serve as a platform for detecting early stage cancerous events and provide early treatment plans for the same.

In one embodiment of this invention, surprisingly, Applicants found that intraluminal administration of the polymers of this invention to a gastrointestinal surface provided for highly sensitive detection. According to this aspect, and in one embodiment, such enhanced sensitivity may therefore provide for early detection of cancerous cells or tissue of gastrointestinal lineage or origin.

For example, such cancerous cells or tissue may include cells or tissue of the digestive, respiratory and reproductive systems.

For example, such cancerous cells or tissue may include esophageal cancer, stomach cancer, gallbladder cancer, gastrointestinal stromal tumors, liver cancer, pancreatic cancer, colon cancer, and other related cancers.

Furthermore, the polymer conjugates are relatively straightforward to prepare, and are associated with reduced costs for synthesis of the same, as compared to other similar diagnostic materials, including polymer conjugates containing peptide-based targeting ligands.

Without being bound by theory, other advantages to the use of the polymer conjugates of this invention may include reduced immunogenicity of the conjugates, reduced toxicity of the conjugates, enhanced stability and shelf-life of the conjugates, enhanced sensitivity of the conjugates as compared to those employing a different polymeric backbone, and other advantages, as will be appreciated by the skilled artisan.

In another embodiment, according to this aspect, the polymers of this invention may be applied intraluminally/applied to other internal mucosal surfaces, for example, within the female reproductive tract, and imaged for early detection of tumors cancerous cells or tissue of female reproductive tissue lineage or origin.

For example, such cancerous cells or tissue may include cervical, ovarian, uterine, vaginal, and vulvar cancer. In some embodiments, such cancerous cells or tissue may include lung cancer.

In some embodiments, according to this aspect, m, n, q and z indicate percentages of the respective monomer composition of the polymer, wherein m is between about 0%-50%, n is between 0.05 to 50%. In some embodiments, m is 0 and the polymer conjugates of this invention contain the polymer and imaging agent alone, no solubilizing agent is included.

In some embodiments, the imaging agent incorporated in the polymer conjugates of this invention, and/or for use in the methods and kits of this invention, is indocyanine green (ICG), or 2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide (IR783).

In some embodiments, the near infrared fluorochromes comprise Cy5.5 and Cy5; IRD41, IRD700, LI-COR and NIR-1, and such agents are commercially available.

The polymer conjugates of this invention may employ spacers, which link the indicated groups to the polymeric backbone. In some embodiments, the spacer arm is an alkane, or in some embodiments, the spacer arm is an alkene or in some embodiments, the spacer arm is a peptidic chain of 6 to 18 atoms, or in some embodiments a combination of such spacers may be incorporated within a given polymeric conjugate of this invention.

Synthesis of the polymer conjugates of this invention may be accomplished by known means.

In some embodiments, with reference to the polymers of this invention, the term “alkane” refers, for example, to branched and unbranched molecules having the general formula CₙH₂ₙ₊₂, wherein n is, for example, a number from 1 to about 100 or more, such as methane, ethane, n-propane, isopropane, n-butane, isobutane, tert-butane, octane, decane, tetradecane, hexadecane, eicosane, tetracosane, and the like Alkanes may be substituted by replacing hydrogen atoms with one or more functional groups. The term “aliphatic” refers, for example, to straight-chain molecules, and may be used to describe acyclic, unbranched alkanes. The term “long-chain” refers, for example, to hydrocarbon chains in which n is a number of from about 8 to about 60, such as from about 20 to about 45 or from about 30 to about 40. The term “short-chain” refers, for example, to hydrocarbon chains in which n is an integer of from about 1 to about 7, such as from about 2 to about 5 or from about 3 to about 4.

In some embodiments, with reference to the polymers of this invention, the term “alkene” refers to any open chain hydrocarbon having carbon to carbon double bonds, wherein each of the carbons containing at least one of the double bonds is joined to either hydrogen or another carbon. Alkenes include compounds having more than one double bond.

In one embodiment, with reference to the polymers of this invention, the alkanes or alkenes may be “substituted”, which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amine, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

In one embodiment “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH₂—NH, CH₂—S, CH₂—S══O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), *-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time. Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

Peptides of this invention may be prepared by various techniques known in the art, including phage display libraries [Hoogenboom and Winter, J. Mol. Biol. 227:381 (1991); Marks et al., J. Mol. Biol. 222:581 (1991)].

In one embodiment, this invention provides a polymer of formula I, III and/or an analog, derivative, isomer, metabolite, pharmaceutically acceptable salt, pharmaceutical product, hydrate, N-oxide, prodrug, polymorph, impurity or crystal or combinations thereof.

In another embodiment, this invention provides a composition comprising a polymer, as described herein.

The invention includes “pharmaceutically acceptable salts” of the polymer of this invention, which may be produced, in one embodiment, using an amino-substituted polymer and an organic and inorganic acids, for example, citric acid and hydrochloric acid. Pharmaceutically acceptable salts can be prepared, from the phenolic compounds, in other embodiments, by treatment with inorganic bases, for example, sodium hydroxide. In another embodiment, esters of the phenolic compounds can be made with aliphatic and aromatic carboxylic acids, for example, acetic acid and benzoic acid esters. As used herein, “pharmaceutically acceptable salt” refers to, in one embodiment, those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M Berge, et al. describe pharmaceutically acceptable salts in detail in J. Pharmaceutical Sciences, 1977, 66: 1-19. The salts can be prepared in situ during the final isolation and purification of the compounds of the invention, or separately by reacting the free base function with a suitable organic acid. Representative acid addition salts include acetate, adipate, alginate, ascorbate, aspartate, benzene-sulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary as ammonium, and mine cations, including, but not limited to ammonium, tetramethylammonium, tetraethylammonium, methylamine, dimethylamine, trimethylamine, triethylamine, ethylamine, and the like.

The invention also includes N-oxides of the amino substituents of the polymer described herein.

This invention provides derivatives of the polymers. In one embodiment, “derivatives” includes but is not limited to ether derivatives, acid derivatives, amide derivatives, ester derivatives and the like. In another embodiment, this invention further includes hydrates of the polymers. In one embodiment, “hydrate” includes but is not limited to hemihydrate, monohydrate, dihydrate, trihydrate and the like.

This invention provides, in other embodiments, metabolites of the polymers. In one embodiment, “metabolite” means any substance produced from another substance by metabolism or a metabolic process.

This invention provides, in other embodiments, pharmaceutical products of the polymers of this invention. The term “pharmaceutical product” refers, in other embodiments, to a composition suitable for pharmaceutical use (pharmaceutical composition), for example, as described herein.

In one embodiment, the polymeric group (P) comprises underivatized or derivatized monomers. In another embodiment, a derivatized monomer refers to a substituted monomer. In another embodiment, the monomer is substituted by an alkyl, halogen, cyano, nitro, amine, phosphonate or any combination thereof. In another embodiment, the monomer is substituted by another monomer forming a copolymer. In another embodiment, derivatized monomer refers to hydrolyzed, oxidized or reduced form of a monomer.

In one embodiment, the polymeric group (P) comprises underivatized or derivatized monomers of N-(2-hydroxypropyl)methacrylamide (HPMA), underivatized or derivatized monomers of N-methylacrylamide, underivatized or derivatized monomers of N,N-dialkylacrylamides, underivatized or derivatized acrylic acid, underivatized or derivatized methacrylic acid polyamino acids, underivatized or derivatized polysaccharides, underivatized or derivatized polymers containing polyethyleneoxide sequences and polyvinyl pyrrolidone-maleic anhydride polymers, underivatized or derivatized polylactic-co-glycolic acid, dendrimers, underivatized or derivatized peptides, underivatized or derivatized proteins, underivatized or derivatized polymer-peptide conjugates or underivatized or derivatized polymer-protein conjugates or mixed polymers.

It is to be understood that P may represent a copolymer of any combination of monomeric units as described in any repeating pattern, or any plausible or desired combination.

In one embodiment, the spacer is selected depending upon the properties desired. For example, the length of the spacer can be chosen to optimize the kinetics and specificity of imaging agent accumulation at cancerous tissue sites. The spacer, in some embodiments, should be long enough and flexible enough to facilitate such accumulation.

In some embodiments, the spacer can be attached to the monomeric units comprising the polymer, using numerous protocols known in the art, such as those described in, for example, Pierce Chemicals “Solutions, Cross-linking of Proteins: Basic Concepts and Strategies,” Seminar #12, Rockford, Ill., and modifications of such methods may be readily achieved, as will be appreciated by the skilled artisan.

In some embodiments, several linkers may be included in order to take advantage of desired properties of each linker Chemical linkers and peptide linkers may be inserted by covalently coupling the linker to the imaging agent, for example. Heterobifunctional agents may be used to effect such covalent coupling. Peptide linkers may also be used.

Flexible linkers and linkers that increase solubility of the polymers are contemplated for use, either alone or with other linkers are also contemplated herein. In some embodiments, such linkers also serve as the solubilizing agents of this invention.

In some embodiments, the solubilizing agents may include methoxy polyethylene glycol (MPEG) and related chemical entities, as will be appreciated by the skilled artisan.

In some embodiments, the solubilizing agents may include an alcohol, propylene glycol, 1,3-butylene glycol, glycerol, polyethylene glycol and derivatives thereof, and mixtures thereof.

The term linker and spacer may, in some embodiments, be considered to be synonymous.

In one embodiment imaging or detection is referred to as radiological. In one embodiment imaging or detection is done by means of an endoscope, for example, as described in Gahlen et al. (1999) J. Photochem. Photobiol. B. 52:131-5; Major et al., 1997, Gynecol. Oncol. 66:122-132, and others. In some embodiments, imaging may be conducted as described herein as part of a hysterosalpingography procedure.

In one embodiment imaging or detection is done by means of a catheter based device, including fiber optics devices, for example, as described in Tearney et al. 1997, Science 276: 2037-2039; Proc. Natl. Acad. Sci. USA 94:4256-4261.

In other embodiments, any appropriate imaging technology may be used, for example, phased array technology (Boas et al. 1994 Proc. Natl. Acad. Sci. USA 91: 4887-4891; Chance 1998, Ann. NY Acad. Sci. 838: 29-45), diffuse optical tomography (Cheng et al., 1998 Optics Express 3: 118-123; Siegel et al. 1999, Optics Express 4: 287-298), intravital microscopy (Dellian et al., 2000, Br. J. Cancer 82: 1513-1518; Monsky et al. 1999 Cancer Res. 59: 4129-4135; Fukumura et al. 1998, cell 94: 715-725) and confocal imaging (Korlach et al. Proc. Natl. Acad. Sci. USA 96: 8461-8466; Rajadhyaksha et al. 1995, J. Invest. Dermatol. 104: 946-952; Gonzalez et al. 1999, J. Med. 30: 337-356), and others as will be appreciated by the skilled artisan.

In another embodiment, the methods of this invention are directed to the imaging of individual cells, a group of cells, a tissue, an organ or a combination thereof.

In one embodiment, imaging is accomplished with computed tomography, computed radiography, magnetic resonance imaging, fluorescence microscopy, angiography, arteriography, or a combination thereof. In one embodiment, a cell is contacted with a polymer of this invention, ex-vivo, and is subsequently implanted in a subject.

In one embodiment, the imaging methods of this invention are conducted on a subject. In another embodiment, the imaging methods are conducted on a sample taken from a subject. In one embodiment, the subject has or is suspected of having cancer.

In one embodiment, the imaging methods as described herein may comprise near infrared fluorescence imaging. In one embodiment, an advantage of such optical imaging methods may include the use of non-ionizing low energy radiation, high sensitivity with the possibility of detecting micron-sized objects, continuous data acquisition, and the development of potentially cost-effective equipment. Optical imaging can be carried out at different resolutions and depth penetrations. Fluorescence-mediated tomography (FMT) can three-dimensionally localize and quantify fluorescent probes in deep tissues at high sensitivity. Several NIR fluorochromes have recently been coupled to affinity molecules (Becker, A., et al. Nature Biotechnology, 19: 327-331, 2001; Folli, S., et al Cancer Research, 54: 2643-2649, 1994, and can be adapted to comprise the polymers of this invention, as will be appreciated by one skilled in the art.

In another embodiment, the polymers of this invention allow for the combination of different imaging modalities.

### Compositions

In one embodiment this invention provides a diagnostic composition comprising the polymers of this invention.

In one embodiment the composition further comprising a carrier, diluent, lubricant, flow-aid, or a mixture thereof. In one embodiment the composition is in the form of a pellet, a tablet, a capsule, a solution, a suspension, a dispersion, an emulsion, an elixir, a gel, an ointment, a cream, an I.V. solution or a suppository.

In one embodiment the composition is in the form of a capsule.

In one embodiment the composition is in a form suitable for oral, intraluminal, intravenous, intraarterial, intramuscular, intracranial, intranasal, subcutaneous, parenteral, transmucosal, transdermal, intratumoral or topical administration.

In some embodiments, the composition and the benefits thereof are particularly suitable for intraluminal administration.

In one embodiment the composition is a controlled release composition. In one embodiment the composition is an immediate release composition. In one embodiment the composition is a liquid dosage form. In one embodiment the composition is a solid dosage form.

In one embodiment the composition further comprises an antineoplastic compound, an immunotherapeutic agent or a drug.

In some embodiments, such compound, an immunotherapeutic agent or a drug may be conjugated to the polymeric backbone. In some embodiments, a concentration of such compound, an immunotherapeutic agent or a drug may be reduced from its recognized therapeutic dose, as a result of enhanced accumulation within target tissue, due to its conjugation to the polymer. In some embodiments, such conjugation may be via a spacer or linker, and via methods as herein described.

In another embodiment, this invention provides a composition comprising a polymer of this invention, which composition further comprising a carrier, diluent, lubricant, flow-aid, or a mixture thereof.

In one embodiment the composition is in the form of a pellet, a tablet, a capsule, a solution, a suspension, a dispersion, an emulsion, an elixir, a gel, an ointment, a cream, an I.V. solution or a suppository. In one embodiment the composition is in the form of a capsule.

Pharmaceutical compositions of this invention for parenteral injection comprise pharmaceutically acceptable sterile aqueous or nonaqueous solutions, dispersions, suspensions, or emulsions as well as sterile powders for reconstitution into sterile injectable solutions or dispersions just prior to use.

Examples of suitable aqueous and nonaqueous carriers, diluents, solvents, or vehicles for general use with the compositions of this invention may include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

In one embodiment the composition is in a form suitable for oral, intraluminal, intravenous, intraarterial, intramuscular, intracranial, intranasal, subcutaneous, parenteral, transmucosal, transdermal, rectally, intracisternally, intravaginally, intraperitoneally, topically (as by powders, ointments, or drops), bucally, or as an oral or nasal spray. The term “parenteral” administration as used herein refers to modes of administration which include intraluminal, intravenous, intramuscular, intraperitoneal, intrathecally, intrasternal, subcutaneous and intraarticular injection and infusion.

In one embodiment the composition can be administered to humans and other animals. In one embodiment the composition is a liquid dosage form. In one embodiment the composition is a solid dosage form. In one embodiment, the compositions of this invention, which comprise a polymer of this invention are biocompatible, and in another embodiment, may comprise pharmaceutically acceptable carriers or excipients, such as disclosed in Remington's Pharmaceutical Sciences, Mack Publishing Company, Easton, Pa., USA, 1985. The polymers, of this invention may be used in the treatment or diagnosis of certain conditions such as in tagging, detecting or removing cancer cells for example from a sample or tissue. These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

The formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions for rectal or vaginal administration are, in one embodiment, suppositories which can be prepared by mixing the compounds of this invention with suitable non-irritating excipients or carriers such as cocoa butter, polyethylene glycol, or a suppository wax which are solid at room temperature but liquid at body temperature and therefore melt in the rectum or vaginal cavity and release the active compound.

The pharmaceutical compositions of the present invention can be used in both veterinary medicine and human therapy. The magnitude of a prophylactic or therapeutic dose of the pharmaceutical composition of the invention will vary with the severity of the condition to be treated and the route of administration. The dose, and perhaps the dose frequency, will also vary according to the age, body weight, and response of the individual patient.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

This invention provides a polymer, which in one embodiment, is water soluble. In one embodiment, water soluble polymers allow for the polymers to be delivered through the blood stream. The polymers of this invention, in some embodiments, offer a number of advantages as delivery systems, as compared to other such systems described in the art, as a result of the unique chemical structure of the polymers of this invention.

The polymers of this invention may assume any structural configuration, which will be a function of, in some embodiments, the chemical makeup of the polymers, and the environment to which the polymer is exposed. In some embodiments, the polymers of this invention may assume a particle configuration.

In some embodiments, through the use of various chain lengths, linkers, side chains, and side chain terminal groups, great flexibility in polymer chemical composition, size, structure, and function can be obtained. In some embodiments, such polymers may be constructed via multiple-step reaction pathways that involve synthesis of a suitable monomer with a protected functional group prior to the polymerization step, followed by deprotection. In other embodiments, the synthesis may be carried out with a chemical/enzymatic/chemo-enzymatic approach as exemplified and described further herein.

Synthesis of the polymer precursors or of the polymers of this invention may be carried out in a number of representative suitable solvents including anhydrous polar aprotic solvents such as acetonitrile, tetrahydrofuran, dioxane, or the like, halogenated solvents such as chloroform, or the like. In some embodiments, synthesis is conducted as exemplified herein, or as a variation thereof, as will be appreciated by the skilled artisan. Synthesis of the monomeric units of the polymers and their linkage to other monomeric units are understood to reflect the choice of monomeric unit and can be accomplished by routine methodology known in the art.

In another embodiment, the polymers are synthesized enzymatically. In one embodiment, the enzymes used to synthesize the polymers of this invention comprise lipases, such as, for example Candida antarctica lipase, or in another embodiment, lipase A, or in another embodiment, lipase B. In another embodiment, the enzyme may comprise an esterase, or in another embodiment, a protease, such as, for example papain or chymotrypsin. In one embodiment, molecular weight of the hydrophilic units is chosen such that its ability to affect polymerization is considered. In one embodiment, the polymer is functionalized with for example, an alkyl group of varying chain length, comprising a polar functionality at the end of the chain.

Polymers obtained by methods as described herein can be characterized by methods well known in the art. For example, the molecular weight and molecular weight distributions can be determined by gel permeation chromatography (GPC), matrix assisted laser desorption ionization (MALDI), and static or dynamic light scattering. Physical and thermal properties of the polymer products can be evaluated by thermal gravemetric analysis (TGA), differential scanning calorimetry (DSC), or surface tensiometer; the chemical structures of the polymers can be determined by, e.g., NMR (1H, 13C NMR, 1H-1H correlation, or 1H-13C correlation), IR, UV, Gas Chromatography-Electron Impact Mass Spectroscopy (GC-EIMS), EIMS, or Liquid Chromatography Mass Spectroscopy (LCMS).

In some embodiments this invention is related to the imaging an inflammatory condition in a subject, the method comprising administering a polymer of this invention, or a composition of this invention to said subject

In one embodiment this invention provides a method of imaging a disease associated with neovascularization in a subject, said method comprising administering a polymer of this invention, or a composition of this invention to said subject.

In one embodiment, this invention provides a method of imaging a cancer or cancerous tissue in a subject, the method comprising the step of contacting a cancer or cancerous tissue with a polymer of this invention, or a composition of this invention.

In one embodiment, the polymer accumulates within tissue containing neoplastic cells.

In one embodiment, the polymers of this invention and/or compositions of this invention are administered orally/luminally to a gastrointestinal tract. In one embodiment, the polymers of this invention and/or compositions of this invention are administered intravaginally, and in one embodiment, the polymers of this invention and/or compositions of this invention are administered via aerosol.

In one embodiment, the polymers of this invention and/or compositions of this invention are administered via any means ensuring application to a mucosal surface

In one embodiment the polymer comprises a spacer. In one embodiment the spacer is (Gly-Gly).

In one embodiment this invention provides a method of diagnosing cancer in a subject, wherein the method comprises contacting a polymer of the present invention to a neoplastic cell or vasculature associated with a neoplastic cell in the subject. In one embodiment the diagnosis comprises the detection of the tag moiety on the polymer. In one embodiment the tag moiety is 2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide. In one embodiment the detection of the tag moiety is an optical detection.

In one embodiment, the term “administering” refers to bringing a subject in contact with the indicated agent. In another embodiment, administration is accomplished in vitro, i.e. in a test tube. In another embodiment, administration is accomplished in vivo, i.e. in cells or tissues of a living organism. Each possibility represents a separate embodiment of the present invention.

In one embodiment cancers are classified by the type of cell that resembles the tumor and, therefore, the tissue presumed to be the origin of the tumor. In one embodiment the cancer type is carcinoma, in which Malignant tumors are derived from epithelial cells. In one embodiment carcinoma represents the most common cancers, including the common forms of breast, prostate, lung and colon cancer. In another embodiment the cancer type is sarcoma. In one embodiment this type of cancer comprises malignant tumors derived from connective tissue, or mesenchymal cells. In another embodiment the cancer type is lymphoma or leukemia. In one embodiment this cancer type comprises malignancies derived from hematopoietic (blood-forming) cells. In another embodiment the cancer type is in the form of a germ cell tumor. In one embodiment such tumor is derived from totipotent cells. In another embodiment, the tumor is a blastic tumor. In one embodiment this is a usually malignant tumor which resembles an immature or embryonic tissue.

In some embodiments, the compounds/compositions and methods of this invention are useful in the diagnosis of any vascularized tumor, for example, a solid tumor, including but not limited to, carcinomas of the lung, breast, ovary, stomach, pancreas, larynx, esophagus, testes, liver, parotid, bilary tract, colon, rectum, cervix, uterus, endometrium, kidney, bladder, prostrate, thyroid, squamous cell carcinomas, adenocarcinomas, small cell carcinomas, melanomas, gliomas, neuroblastomas, sarcomas (e.g., angiosarcomas, chondrosarcomas).

In some embodiments, the compounds/compositions and methods are useful in diagnosing other diseases associated with neovascularization, such as, but not limited to inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells

Other angiogenesis-associated diseases or disorders which can be diagosed with the compounds/compositions or by the methods encompassed by the present invention include, but are not limited to, osteoarthritis, lupus, systemic lupus erythematosis, polyarteritis, artery occlusion, vein occlusion, carotid obstructive disease, sickle cell anemia, pseudoxanthoma elasticum, Paget's disease, lyme's disease, Best's disease, Eale's disease, Stargardt's disease, toxoplasmosis, phylectenulosis, lipid degeneration, chronic inflammation, atherosclerosis, hereditary diseases, such as Osler-Weber-Rendu disease.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

The following examples are presented in order to more fully illustrate some embodiments of the invention. They should, in no way be construed, however, as limiting the scope of the invention.

### Example 1

### Synthesis of Polymer Conjugates

### Synthesis of IR-783 Dye with a Free Carboxylic Acid Group (IR-783-S-pH-COOH)

IR-783-S-Ph-COOH was synthesized based on a previously described procedure (Wang et al., Bioconjugate Chem., Vol. 18, No. 2, 2007) (see scheme 1 below). Briefly, IR-783 was conjugated with 4-mercaptobenzoic acid in DMF in the presence of DIPEA at 1:1:6 molar ratio. The mixture was stirred over night. The solvent was evaporated and the product was purified by silica gel column, mobile phase ethylacetate:methanol (1:1) and analyzed by MALDI. Yield: 92%,

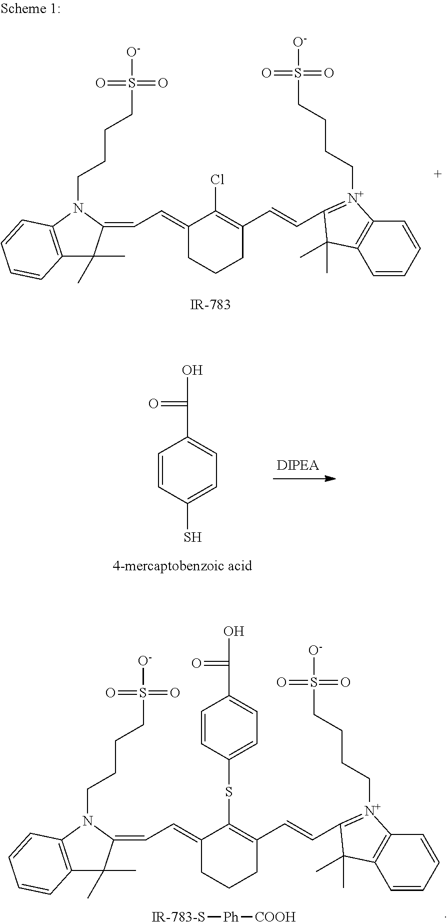

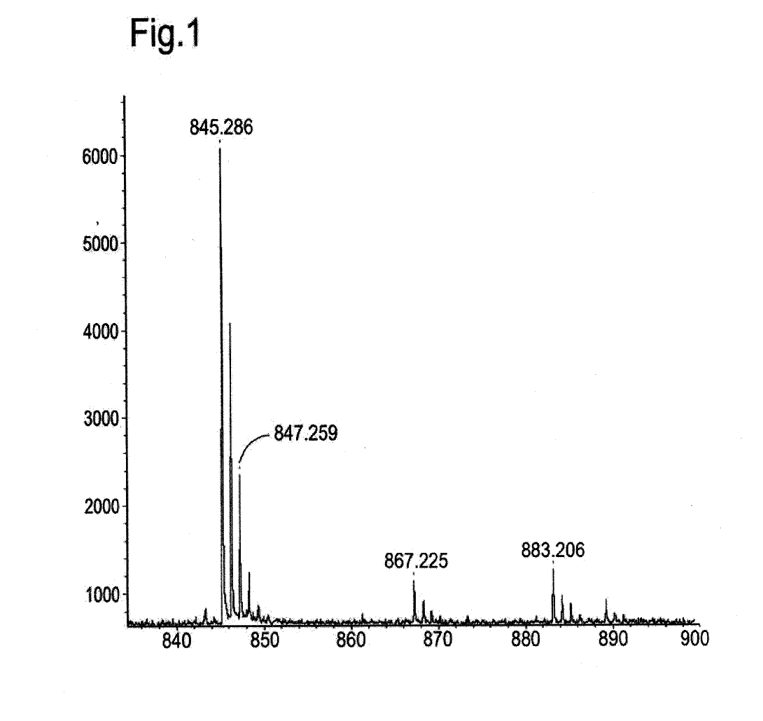

FIG. 1 depicts the MALDI-TOF mass spectrometry results showing peaks at 845.3, 867.2 and 883.2, calculated for M+H+, M+Na+, and M+K+, respectively.

### Synthesis of Synthesis of HPMA Copolymer Precursor for IR783-S-pH-COOH Attachment P-(GG-ONp)-(AP-Boc)

An HPMA copolymer precursor having aminopropyl-side chains for IR-783-S-Ph-COOH attachment (designated as P-(GG-ONp)-(AP-Boc), where P represents the HPMA copolymer backbone) was synthesized by random radical precipitation copolymerization in a sealed vial in acetone/DMSO mixture at 50° C. for 24 hr using AIBN as the initiator. The feed molar percentage of the monomers was 84.5:8:7.5 for N-(2-hydroxypropyl)methacrylamide (HPMA), methacylolyl-glycyl-glycine-O-nitrophenyl (MA-GG-ONP) and 3-aminopropyl methacrylamide (MA-AP-Boc), respectively. The ratio of monomers to initiator and solvent was 12.5:0.6:86.9 wt %, respectively. The content of the monomers in the copolymer was calculated by H¹-NMR.

The M_{w}, Mₙ and P_{I} of the polymers were determined as described in size exclusion chromatography on Fast Protein Liquid Chromatography (FPLC) system using Sephacryl 16/60 S-400 column with PBS buffer, calibrated with fractions of known molecular weight HPMA copolymers. The molar percentage of MA-AP-Boc monomer was assessed by ¹H-NMR at 500 Hz in D₂O, using the Boc t-butyl protons chemical shift (d 1.40, s, 9H)

### Synthesis of HPMA Copolymer Bearing IR783-S-pH-COOH (P-(AP)-IR783)

The precursor copolymer P-(GG-ONp)-(AP-Boc) (25 mg) was first dissolved in NaOH/DDW to remove the ONp groups and then dissolved in TFA for 8 min to remove the Boc protecting group to yield a precursor copolymer with free amine groups, designated P-(GG-OH)-(AP-NH₂). The solution was concentrated by evaporation, and the polymers were precipitated in cold ether, and dried.

The polymer P-(GG-OH)-(AP-NH2) was dissolved in dry DMF and coupled with IR-783-S-Ph-COOH dye (42 mg), that was pre-activated for 3 min with HBTU (19 mg) and DIPEA (5 μL) coupling reagents. The reaction was stirred overnight at RT, precipitated in acetone: ether (1:1) dried and purified using PD-10 column. The M_{w}, Mₙ and P_{I} of the polymers (P-(AP)-IR783) were determined as described above. Molar percentage of IR-783-S-Ph-COOH was determined spectrophotometrically.

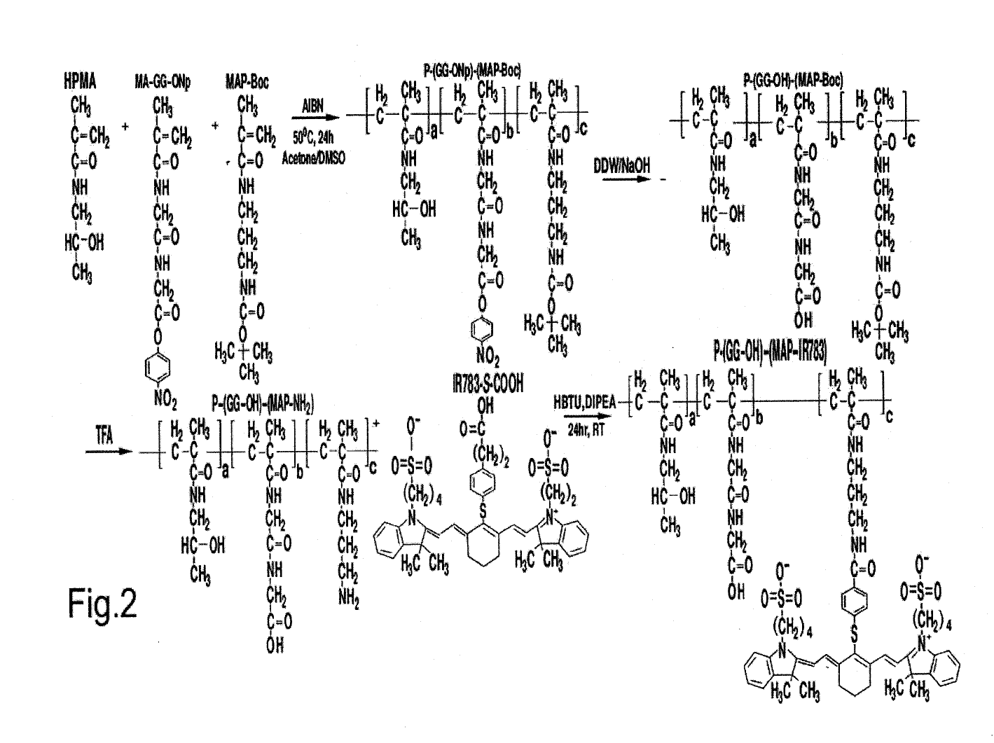

FIG. 2 schematically depicts the synthetic scheme described herein.

### Example 2

### Selective Accumulation of Embodied Polymer-Conjugates within Cancerous Tissue Following Intra-Luminal Administration

### Intraluminal Administration of the Polymer Conjugates to Mice Harboring Tumors in LS174T and HT29 Models

A 9.2% solution of P-(AP-IR783) in PBS was prepared according to Example 1 and administered intracolonically by colonoscopy to female athymic nude mice bearing rectal tumors following LS174T and HT29 cell injections. 4-week old lumen-facing LS174T tumors were anaesthetized and treated with P-(AP-IR783) solution in PBS (0.2 mg/ml), applied intracolonic with the guidance of a mini colonoscopy. 20 min later the colon was washed extensively with PBS and then were allowed to recover for 3 h. Then, the mice were sacrificed and the colons were removed. Each colon was spread on a clear film, and imaging was performed using the Odyssey® Infrared Imaging System (Li-Cor Biosciences, Lincoln, Nebr., USA.), with excitation wavelength of 780 nm and emission wavelength of 800 nm Results:

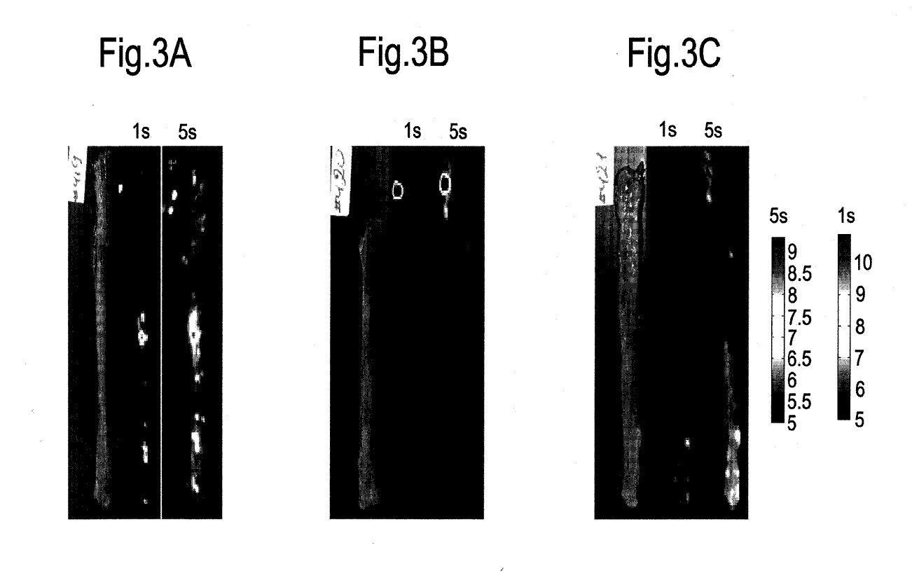

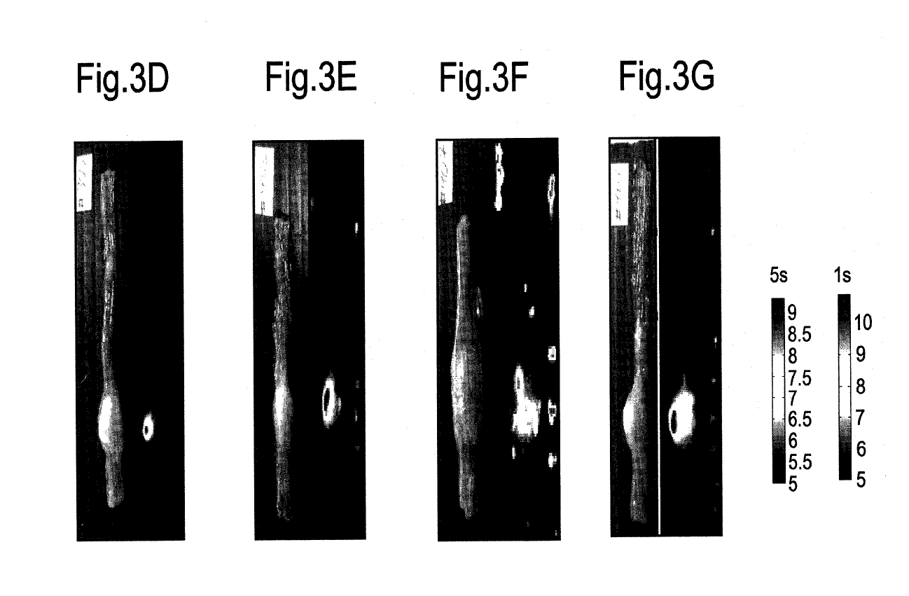

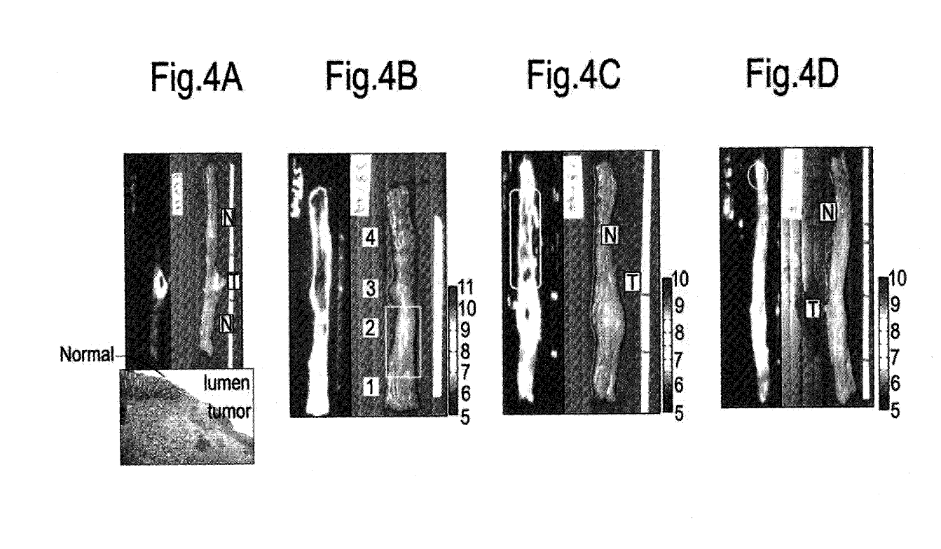

Selective accumulation of P-(AP-IR783) in cancerous tissue occurred when the polymer was applied intraluminally to mice in LS174T and HT-29 tumor models (FIG. 3-4). When 40 μg per mouse (0.2 mg/ml) of P-(AP-IR783) was applied to control mice without tumors (FIG. 3A-C), very little background staining is seen (images taken at 4 hours following washing). In contrast, clear staining was evident in samples taken from mice having tumors (verified by pathologic evaluation subsequently) (FIGS. 3D-G). Tumor associated-vasculature staining was evident, as well (FIG. 3F), this despite the intraluminal application, noting efficient uptake of the polymer by the cancerous tissue.

A similar study was conducted using 0.1 mg/ml (200 μg) P-(AP-IR783) applied and animals were visualized only 2 hours after the wash (FIG. 4A).

In another tumor model, when mouse HT-29 models were evaluated immediately post administration of 0.1 mg/ml (200 μg) P-(AP-IR783), surface exposed regions (boxes 2, 3) were stained intensely, but whereas region 4 did not evidence the presence of tumors when viewed macroscopically, the region stained intensely, and proved to contain tumors in the submucosa (FIG. 4B). FIGS. 4C and 4D provide results of similarly treated animals, 2 hours post-administration. The phenomenon of evident staining of both surface exposed (T) and submucosal tumors (N) is maintained even two hours after exposure, indicating retention/accumulation of the marker in cancerous tissue.

### Example 3

### In Situ Labeling Experiments in Human Colorectal Cancer Tissues

### Application of the Polymer to Human Colorectal Cancer Biopsy Specimens

An aqueous solution (PBS, 0.2 mg/ml) of 9.2% P-(AP-IR783) prepared according to Example 1 was applied to surgically excised cancerous colorectal tissue, obtained from 3 patients [male] by informed consent at the Belinson Medical Center. Polymer solution was dropped onto fresh surgical tissue specimens that were received 15 minutes after surgical excision. After 20 minutes of incubation with the polymeric probe, tissues were washed three times with a large volume of PBS. Tissues were then imaged immediately using the Odyssey® Infrared Imaging System (Li-Cor Biosciences, Lincoln, Nebr., USA.) with excitation wavelength of 780 nm and emission wavelength of 800 nm.

### Results

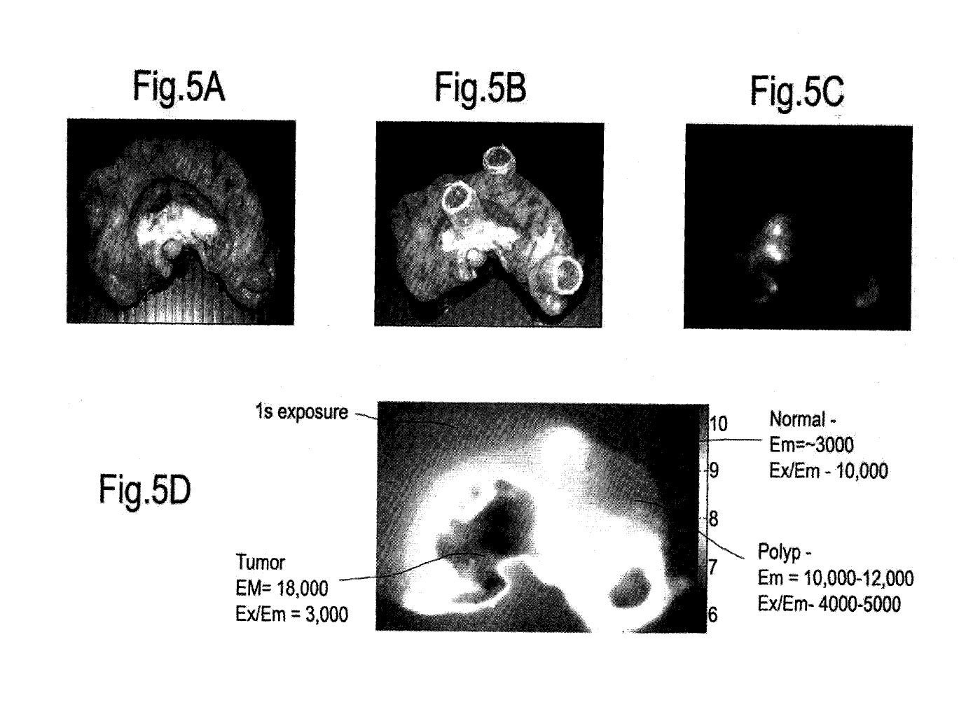

FIG. 5 presents selective markedly intense staining of the applied conjugate polymer at cancerous region in the tissue (polyp and tumor) with very low binding to near healthy tissue. FIG. 5B depicts the administration protocol in that the polymeric solution was applied to the open “cups” evident in the photograph, and the arrangement of the 2-sided open cups ensured that the solution remained at the site for at least 20 minutes post application.

### Example 4

### Selective Accumulation of Embodied Polymer-Conjugates within Cancerous Tissue Following Intra-Luminal Administration

The murine orthotopic colorectal tumor model LS-174T human colorectal adenocarcinoma cells (3×10⁷ cells) were injected (600 μL) into the descending colonic wall of 13 anesthetized, female athymic nude mice, after an over night fast. As a result, the carcinoma developed on the serosal (abdominal) surface of the intestine without invading the mucosa, causing its irregular thickening, but did not expand to grow over the mucosa (i.e., did not form a protruding nodule). Histopathological examination was performed on paraffin fixed colonic tissue specimens, after an H&E stain and verified that the carcinoma infiltrated within the mucosa, submucosa, and muscular layer

Thirty days after tumor induction, aqueous solution (PBS, 0.2 mg/ml) of 9.2% of P-(AP-IR783) prepared according to Example 1 was instilled into the colon of the anesthetized mice over 20 minutes, after which colons were washed ×3 times with large volumes of PBS. The mice were sacrificed 3 hrs post treatment, their colons exteriorized, separated, cut open, spread on a transparent film with the mucosal aspects upwards, and imaged by the Odyssey® Infrared Imaging System (Li-Cor Biosciences, Lincoln, Nebr., USA.) with excitation wavelength of 780 nm and emission wavelength of 800 nm.

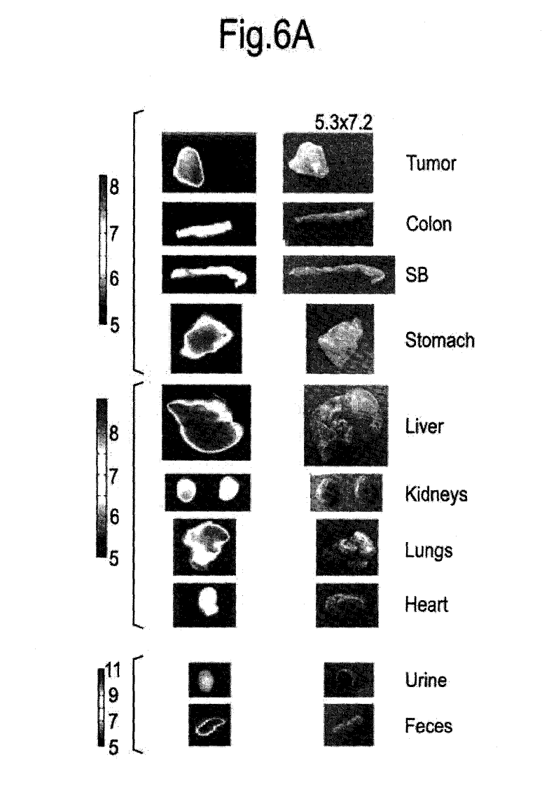

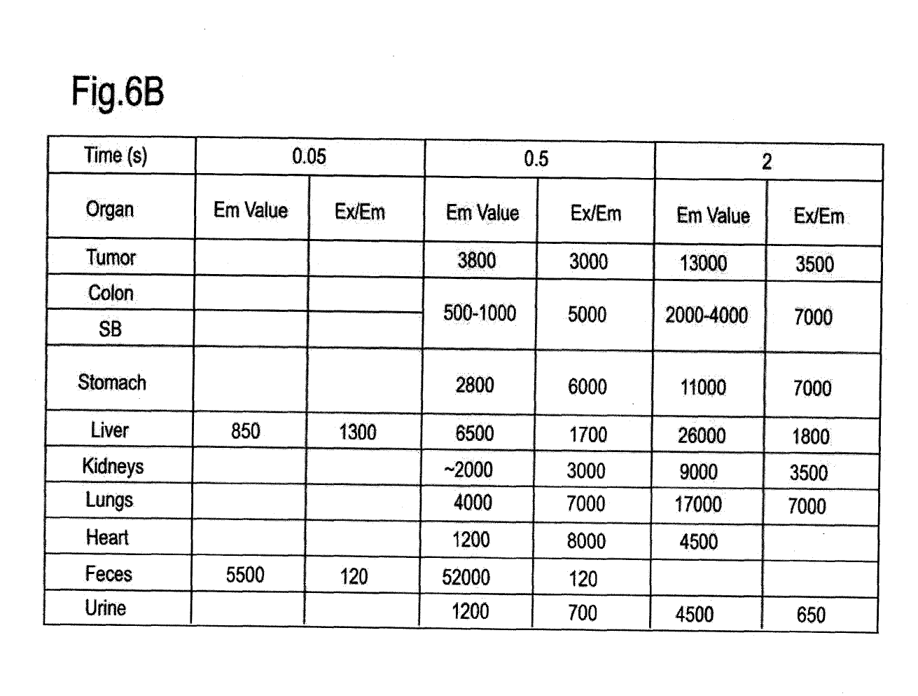

Selective accumulation of P-(AP-IR783) in cancerous tissue occurred when the polymer was applied intraluminally to the mice (FIG. 6). When 40 μg per mouse (0.2 mg/ml) of P-(AP-IR783) was administered intraluminally, intense staining was observed in the excised tumor colon tissue, stomach, liver, lungs and feces (FIG. 6A), with very little background staining is seen in unaffected tissues and samples (e.g. heart, small bowel, urine) (images taken at 3 hours following washing). The table in FIG. 6B plots the significance of these findings in terms of the Em value obtained at 0.05, 0.5 and 2 seconds post administration [the indicated times 0.05, 0.5 and 2, represents the exposure time] showing staining over time in the stomach, liver and lungs. Tumor associated-accumulation was therefore selectively evident, in perfused tissues which possess cancerous tissue, but accumulation was not found in irrelevant, well perfused tissue such as the heart or in the urine.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A polymer characterized by the structure of formula 1: 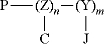
wherein
m, n and indicate percentages of the respective monomer composition of the polymer, wherein m is between about 0%-50%, n is between 0.05 to 50%;
C is a near infrared dye selected from the group consisting of Cy5, Cy5.5, Cy7,
Indocyanine green (ICG), IR783 and analogs thereof, covalently linked to the polymeric backbone and present in a concentration of between about 0.5 to 50%.
Y is a spacer arm linking J to the polymeric backbone, wherein said spacer arm is an alkane, alkene or a peptidic chain of 6 to 18 atoms;
Z is a spacer arm linking C to the polymeric backbone, wherein said spacer arm is an alkane, alkene or a peptidic chain of 6 to 18 atoms
J is a solubilizing agent and present in a concentration of between about 0 to 50%, and
P is a polymeric backbone comprising underivatized or derivatized N-(2-hydroxypropyl)methacrylamide (HPMA) monomers, underivatized or derivatized N-methylacrylamide monomers, underivatized or derivatized N,N-dialkylacrylamides monomers, underivatized or derivatized acrylic acid, underivatized or derivatized methacrylic acid polyamino acids, underivatized or derivatized polysaccharides, underivatized or derivatized polymers containing polyethyleneoxide sequences and polyvinyl pyrrolidone-maleic anhydride polymers, underivatized or derivatized polylactic-co-glycolic acid, dendrimers, underivatized or derivatized peptides, underivatized or derivatized proteins, underivatized or derivatized polymer-peptide conjugates or underivatized or derivatized polymer-protein conjugates or mixed polymers.

2. The polymer of claim 1, wherein Y is characterized by the structure of formulae IIa, or IIb or IIc as follows: 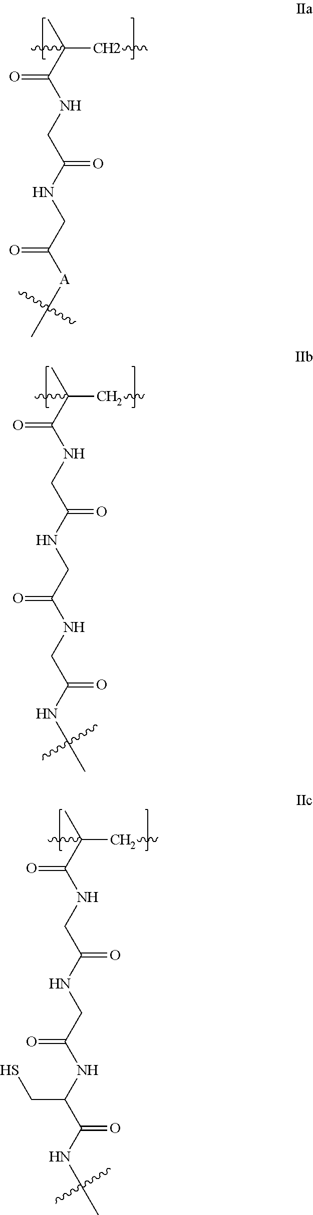
where A is an amine or an alcohol.

3. The polymer of claim 1, wherein the molecular weight of said polymer ranges between 15-60 kDa.

4. The polymer of claim 1, wherein said polymer is water soluble.

5. The polymer of claim 1, wherein said imaging agent is 2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide.

6. The polymer of claim 1, wherein said polymer is represented by the structure of formula III: 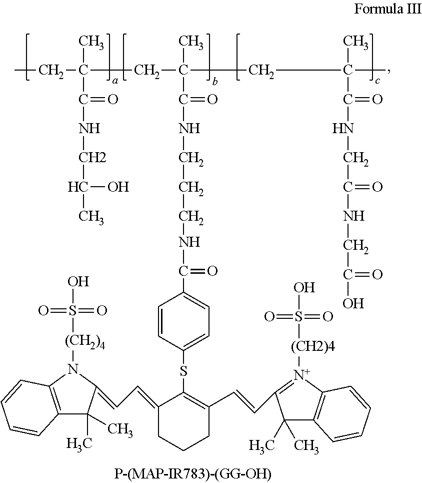
wherein a, b, c and indicate percentages of the respective monomer composition of the polymer,
wherein b is between 0.05 to 50%, a and c is between 0 to 50%.

7. A diagnostic composition comprising the polymer of claim 1.

8. A method of imaging an inflammatory condition in a subject, said method comprising administering a polymer of claim 1 to said subject.

9. A method of imaging a disease associated with neovascularization in a subject, said method comprising administering a polymer of claim 1 to said subject.

10. A method of imaging cancer cells or cancerous tissue in a subject, said method comprising the step of contacting said cancer cells or cancerous tissue with a polymer of claim 1.

11. The method of claim 10, wherein said polymer preferentially accumulates proximally to neoplastic cells or tissue.

12. The method of claim 10, wherein, said cancer cells are derived from the lung, breast, prostate, colon, or pancreas.

13. The method of claim 10, wherein said neoplastic cells are carcinoma, sarcoma, lymphoma, or leukemia cell.

14. The method of claim 10, wherein said method is used to image cancerous tissue of the colon or colorectal cancerous tissue

15. The method of claim 10, further comprising the step of providing anti cancer therapy to imaged cancer or cancerous tissue in said subject.

16. The method of claim 15, wherein said anti-cancer therapy comprises surgery, chemotherapy, radiation or a combination thereof.

17. The method of claim 10, wherein said method further comprises establishing a diagnosis based on the detection of said tag moiety on said polymer.

18. The method of claim 17, wherein said detection of the tag moiety is an optical detection.

19. The method of claim 9, wherein said polymer is administered intraluminally to the gastrointestinal tract.

20. The method of claim 10, wherein said polymer is administered intraluminally to the gastrointestinal tract.
